(19) United States
(12) Patent Application Publication
(10) Pub. No.: US 2025/0366975 A1
(43) Pub. Date: Dec. 4, 2025

(54) SYSTEMS AND SUPPORT STRUCTURE FOR TREATING AN ANEURYSM

(71) Applicant: Life Seal Vascular, Inc., Costa Mesa, CA (US)

(72) Inventors: Michel Marinus Petrus Johannes Reijnen, Arnhem (NL); William Colone, Costa Mesa, CA (US)

(73) Assignee: Life Seal Vascular, Inc., Costa Mesa, CA (US)

(21) Appl. No.: 18/862,907

(22) PCT Filed: Apr. 25, 2023
(86) PCT No.: PCT/US2023/019837

§ 371 Date: Nov. 4, 2024

Related U.S. Application Data
(60) Provisional application No. 63/334,637, filed on Apr. 25, 2022.

(51) Int. Cl. A61F 2/07

(52) U.S. Cl. CPC A61F 2/07; A61F 2002/072; A61F 2002/077

(57) ABSTRACT

Systems and a support structure for treating an aneurysm are provided. A system includes a stabilizing structure that is configured to surround a lumen structure that facilitates blood flow through the aneurysm where the support structure includes one or more elements that are configured to expand to fill a space in an aneurysmal sac.

### CROSS REFERENCE TO PRIOR APPLICATION

This application claims the benefit of U.S. Provisional Patent Application No. 63/334,637, entitled as “SYSTEMS AND SUPPORT STRUCTURE FOR TREATING AN ANEURYSM”, filed Apr. 25, 2022, which is incorporated by reference in its entirety.

### FIELD OF THE INVENTION

This disclosure relates to the field of treatment for aneurysms.

### BACKGROUND

Stent grafts are a common treatment for aortic aneurysms in general, whereby a stent graft is inserted into the aneurysms to facilitate the transport of blood through the aneurysm from the aortic artery to the iliac arteries, thereby depressurizing the aneurysm, and preventing aneurysm rupture. An endoleak is a leakage of blood into the space in the aneurysmal sac outside the stent graft, which may be referred to as the excluded aneurysm sac. The leakage of blood into the excluded aneurysm sac is classified into five types.

A type I endoleak occurs where blood leaks through at least one of the attachment sites at the aortic artery or iliac arteries. Type I endoleaks carry a high risk of aneurysm sac rupture because they tend to be high pressure. A type II endoleak occurs where blood flows into the excluded aneurysm sac through open collateral arteries. Type II endoleaks are lower pressure than type I and type III endoleaks. Type III endoleaks occur where blood leaks through a body of the stent graft into the excluded aneurysm sac. Type IV endoleaks occur due to porosity of the graft materials. Type V endoleaks occur where the aneurysmal sac continues to grow without direct evidence of a leak. Although all types of endoleaks can become serious, type I and type III endoleaks generally require urgent medical attention. There is a need in the art for solution that mitigates the risk of endoleaks and thus reduces a need for medical attention subsequent to installation of a stent graft.

### SUMMARY

The disclosed subject matter comprises systems and a support structure for treating an aneurysm, in adjunct to a conventional stent graft, to prevent endoleaks and promote aneurysm sac regression, thereby reducing reinterventions. An exemplary embodiment is a system for treating an aneurysm. The system includes a stabilizing structure that is configured to surround a lumen structure that facilitates blood flow through the aneurysm where the support structure includes one or more elements that are configured to expand to fill a space in an aneurysmal sac. At least one of the one or more elements may include a sheet that at least partially wraps around the lumen structure. At least two of the one or more elements may include a sheet or a tube that at least partially wraps around the lumen structure where the at least two elements wrap around the lumen structure in layers. Lengths. as measured along a flow of blood in the lumen structure, of the two or more elements are tapered such that the lengths decrease from an inside layer to an outside layer. Tapering of the layers is configured to fit within the space in the aneurysmal sac. The stabilizing structure may prevent displacement of the lumen structure within the aneurysm. At least one of the one or more elements may include a longitudinal element. At least one of the one or more elements may further include a longitudinal element where the longitudinal element is configured to be inserted in a space of the aneurysmal sac outside the at least one sheet or a tube that at least partially wraps around the lumen structure. The stabilizing structure may include a multitude of longitudinal elements. The stabilizing structure may lower a risk of endoleak compared to the lumen structure without the stabilizing structure. The endoleak may comprise a type I, type II, type III, or type IV endoleak. The one or more elements may include a foam material. The one or more elements may be configured to be delivered to the aneurysmal sac prior to delivery of the lumen structure. The sheets or tubes may be configured to be delivered in an order from the outmost layer to the innermost layer.

Another general aspect is a support structure for a stent graft. The support structure includes one or more elements that are configured to expand to fill a space in an aneurysmal sac where at least one of the one or more elements comprises a sheet or tube that at least partially wraps around the stent graft. The one or more elements may concentrically wrap around the stent graft in layers where the one or more elements comprise at least two elements and lengths of the one or more elements, as measured along a flow of blood in the lumen, of the two or more sheets are tapered such that the lengths decrease from an inside layer to an outside layer. The at least two elements may be configured to be delivered in an order from the outermost layer to the innermost layer. The one or more elements may be configured to be delivered prior to delivery of the stent graft.

An exemplary embodiment is a system for treating an aneurysm. The system includes an endograft comprising an interior lumen that facilitates blood flow across an aneurysmal sac where one or more elements are configured to expand to fill a space in the aneurysmal sac between the endograft and interior walls of the aneurysmal sac. At least one of the one or more elements includes sheets or tubes that at least partially wrap around the endograft. The sheet may wrap around the endograft in layers. The one or more elements may be configured to be delivered to the aneurismal sac in an order from an outermost layer to an innermost layer. Lengths, as measured along a flow of blood in the interior lumen, of the one or more elements may be tapered such that the lengths decrease from an inside layer to an outside layer. The one or more elements may be configured to be delivered through an opening of a catheter that comprises a dilator.

### BRIEF DESCRIPTION OF THE DRAWINGS

FIG. 1 is an illustration of the abdominal aortic artery, renal arteries, and bifurcation into the iliac arteries with representations of changes in the abdominal aortic artery during an aneurysm.

FIG. 2 is an illustration of a stent graft that has been implanted to treat an abdominal aortic aneurysm.

FIG. 3 is an illustration of a stent graft surrounded by multiple layers of a stabilizing structure within the excluded aneurysm sac.

FIG. 4 is an illustration of a cross section of multiple layers of a stabilizing structure and thrombi within the excluded aneurysm sac.

FIG. 5 is an illustration of a delivery system for delivering the stabilizing structure to the aortic aneurysm.

FIG. 6 is an illustration of a tapered layering of the stabilizing structure without environmental features such as an aneurysm, blood vessels, or a stent graft.

FIG. 7 is an illustration of the tapered layering of the stabilizing structure after being deployed in an aneurysm and before deployment of a stent graft.

FIG. 8 is a photograph of multiple layers of a stabilizing structure from a top view.

FIG. 9 is a photograph of multiple layers of a stabilizing structure from a top perspective view.

FIG. 10 is a photograph of a sheet of material used as the stabilizing structure from a perspective view.

FIG. 11 is a photograph of a single layer of a curved material used as the stabilizing structure from a top view.

FIG. 12 is a photograph of a single layer of a curved material used as the stabilizing structure from a top view.

FIG. 13 is a photograph of a single layer of a circular portion of the stabilizing structure from a perspective view.

FIG. 14 is a photograph of multiple layers of a stabilizing structure apart from one another from a top view.

FIG. 15 is a photograph of an embodiment of the stabilizing structure comprising pellets of material to be inserted into the excluded aneurysm sac.

### DETAILED DESCRIPTION

The disclosed subject matter is a stabilizing structure to prevent or diminish a severity of endoleaks subsequent to deployment of a stent graft to an aneurysm. An exemplary embodiment of the disclosed subject matter comprises a stabilizing structure configured to surround a stent graft for an abdominal aortic aneurysm. In various embodiments, the stabilizing structure is configured to be positioned in the excluded aneurysm sac. There, the stabilizing structure may provide a stabilizing force for a stent graft and/or impede a flow of blood into the excluded aneurysm sac.

In various embodiments, the stabilizing structure is a porous spongelike material such as a foam. An exemplary embodiment of the stabilizing structure comprises a resilient foam material that expands from a compressed state into an uncompressed state without permanent change to the structure of the foam material. In various embodiments, the stabilizing structure is delivered to a position in an abdominal aortic aneurysm while the stabilizing structure is in a compressed state. After delivery, the stabilizing structure may expand into an uncompressed state or until the stabilizing structure contacts an inner barrier withing the abdominal aortic aneurysm. The inner barrier may be an inner lumen of the abdominal aortic aneurysm, a thrombus, another stabilizing structure, another medical device, or the like.

In various embodiments, the stabilizing structure comprises 2 or more layers. The layers of the stabilizing structure may each comprise a sheet of spongelike material. The sheet of spongelike material may at least partially wrap around an outer perimeter of the abdominal aortic aneurysm such that the stabilizing structure leaves space for a stent graft in the center of the aneurysm. In various embodiments, the 2 or more layers comprise concentric tubes. Each of the layers of the stabilizing structure may be delivered in sequence where the outermost sheets are delivered to a position in the abdominal aortic aneurysm before layers that are positioned inside the layer. For example, in a stabilizing structure comprising 3 concentric layers, an outermost layer may be delivered first followed by a second middle layer. The inner layer is delivered last.

In various embodiments, layers of the stabilizing structure may be delivered in a compressed state by a catheter. The layers of the stabilizing structure may be held in the compressed state by various means, including through compressive force from the inner walls of the catheter. In an exemplary embodiment, the stabilizing structure may be pushed out of the catheter at the delivery location by a plunger. Further, the catheter may include a dilator that comprises a cone on a distal end of the catheter that increases in radius relative to the catheter from a proximal end of the dilator to a distal end of the dilator. The dilator may facilitate expansion of the layers of the stabilizing structure from a compressed state to an expanded state by allowing each layer to gradually expand as it is pushed through the dilator.

In various embodiments, each of the layers of the stabilizing structure has a different length as measured along an axis from the upper portion of the abdominal aortic artery to the bifurcation of the abdominal aortic artery into the iliac arteries. The varied lengths may be configured to fit the shape of the aortic aneurysm. For example, where the stabilizing structure has 3 layers, the 1^{st} (outer) layer, which is positioned on the outside of the stabilizing structure, has the shortest length. The 2^{nd} (middle) layer may have a longer length than the 1^{st} layer and the 3^{rd} (inner) layer may have the longest length of the 3 layers. In various embodiments, the lengths of each of the one or more layers may be tailored to fit the dimensions of the abdominal aortic aneurysm for each individual patient.

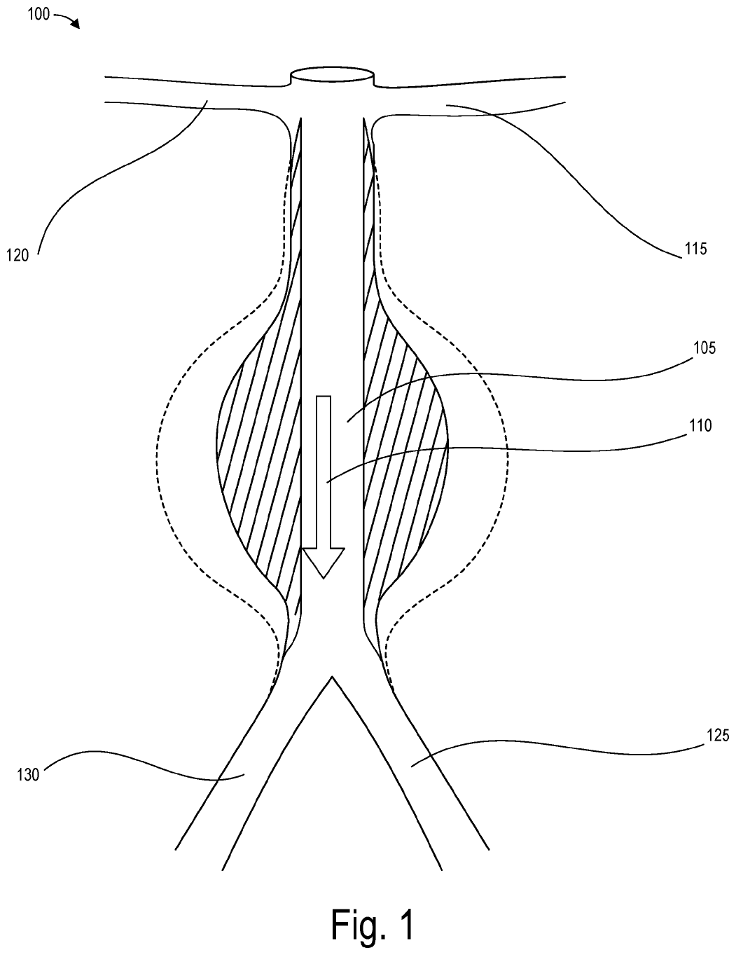

Referring to FIG. 1, FIG. 1 is an illustration 100 of the abdominal aortic artery, renal arteries, and bifurcation into the iliac arteries with representations of changes in the abdominal aortic artery during an aneurysm. In the perspective shown in the illustration 100, the aortic artery 105 pumps blood from the heart in a downward direction 110. The left renal artery 115 and right renal artery 120 branch from the aortic artery 105, as shown in the upper portion of the illustration 100. The lower portion of the illustration 100 shows the bifurcation of the aortic artery 105 into left iliac artery 125 and right iliac artery 130. There are multiple collateral vessels with access to the aortic artery 105 between the renal arteries and iliac arteries. The most common collateral vessels are the lumbar arteries and mesenteric arteries.

An abdominal aortic aneurism is an expansion of the aortic artery 105. It usually occurs in the portion of the aortic artery 105 between the renal arteries and the bifurcation into left iliac artery 125 and right iliac artery 130. The expansion of the aortic artery can potentially result in a rupture, which is a life-threatening condition.

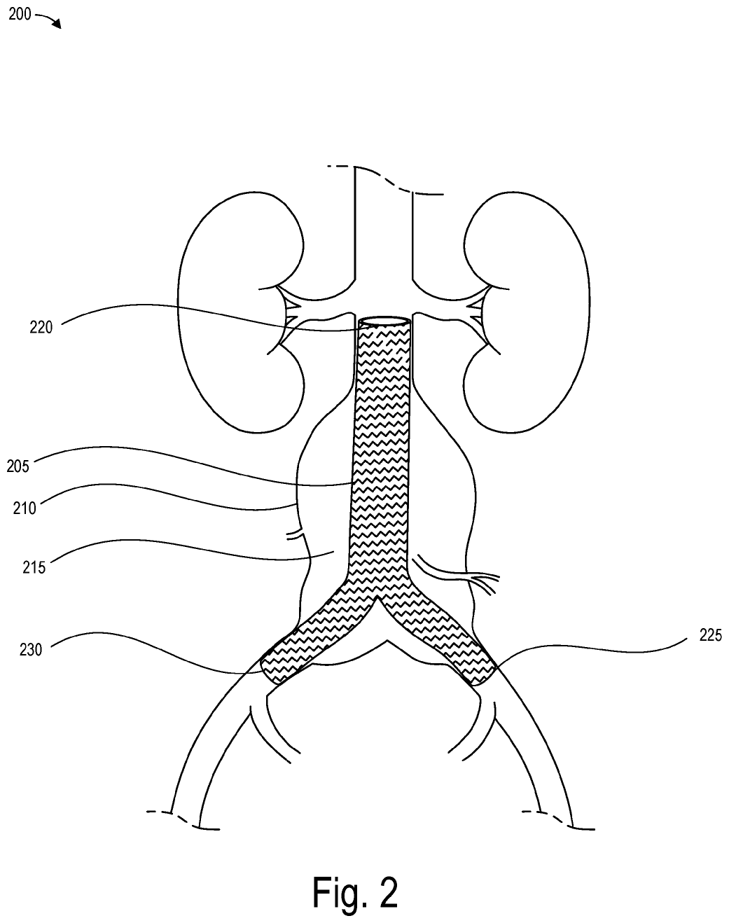

Referring to FIG. 2, FIG. 2 is an illustration 200 of a stent graft 205 that has been implanted to treat an abdominal aortic aneurysm 210. The space of the abdominal aortic aneurysm 210 in between the stent graft 205 and the inner wall of the abdominal aortic aneurysm 210 may be referred to as the excluded aneurysm sac 215. An endoleak, whereby blood fills the excluded aneurysm sac 215, may lead to expansion of the abdominal aortic aneurism and potentially lead to a life-threatening rupture.

There are multiple types of endoleaks depending on the path that blood takes to enter the excluded aneurysm sac 215. When blood enters the excluded aneurysm sac 215 through one of the attachment points of the stent graft to the blood vessels, it is considered a type I endoleak. For instance, blood may enter the excluded aneurysm sac 215 through the attachment point 220 of the stent graft 205 to the aortic artery where blood enters the stent graft 205. Blood may also enter the excluded aneurysm sac 215 through the left iliac artery attachment point 225 or right iliac artery attachment point 230 to the stent graft 205.

When blood enters the excluded aneurysm sac 215 through one of the collateral vessels such as the mesenteric artery or lumbar artery, it is considered a type II endoleak, which is not as serious as a type I endoleak, but could require medical attention nonetheless. When blood enters the excluded aneurysm sac 215 through a defect in the material of the stent graft 205, it is considered a type III endoleak. Type I and III endoleaks can result in high pressure inside the aneurysm that can quickly lead to a rupture of the abdominal aortic aneurysm.

When blood enters the excluded aneurysm sac 215 through pores in the material of the stent graft 205, it is considered a type IV endoleak. Type IV endoleaks typically correct after a period of time and are usually not serious. And when blood enters the excluded aneurysm sac 215 through an unknown pathway, it is considered a type V endoleak, which is rare.

The disclosed stabilizing structure helps to prevent movement of the stent graft 205, which could potentially result type I or type III endoleaks. Further, the disclosed stabilizing structure applies a constant pressure to open spaces in the excluded aneurysm sac 215, which could potentially mitigate blood flow into the excluded aneurysm sac 215 for all types of endoleaks.

An artificial lumen, or lumen structure, facilitates a flow of blood through a blood vessel that may be compromised such as through the aneurysm shown in the illustration 200. There are many variations of lumen structures to which the disclosed subject matter may be applied. One type of lumen structure is a stent graft 205, which comprises a mesh tube (stent) surrounded by material (graft).

An example of a stent is a mesh that is configured to be delivered to a blood vessel in a compressed state. Once delivered, the stent may be caused to expand by various means into a tube shape that approximates a blood vessel. Various stent materials comprise a metal material, such as nitinol, that expands into a predefined shape when exposed to blood after being delivered by a catheter. Other stent meshes, such as those made with stainless steel, are expanded manually via balloons or push wires.

The graft material may be any material that surrounds the stent mesh and impedes blood from traversing the walls of the graft material. The inside surface of the graft material creates the inner lumen of the lumen structure and encounters blood that flows through it. The outside surface of the graft structure contacts the disclosed stabilizing structure for the aneurysm. Various graft materials include but are not limited to polyester, polytetrafluoroethylene (PTFE), and polyethylene terephthalate (PET). The graft material is typically attached to the stent prior to delivery of the stent graft. Thus. the graft is compressed or folds with the stent during delivery and expands or unfolds as the stent expands within the blood vessel.

The stent graft 205 may be anchored within the blood vessel to prevent movement or migration of the stent graft 205. In particular, the attachment points of the stent graft 205. where blood enters and exits the stent graft 205, should be secure to the walls of the blood vessel to prevent leakage of the blood to the damaged portion of the blood vessel. Anchoring may be accomplished by various means including but not limited to mechanical pressure from the expanded stent against the inner lumen of the blood vessel. barbs in the stent graft 205 that pierce the blood vessel, and another medical device that provides an anchor.

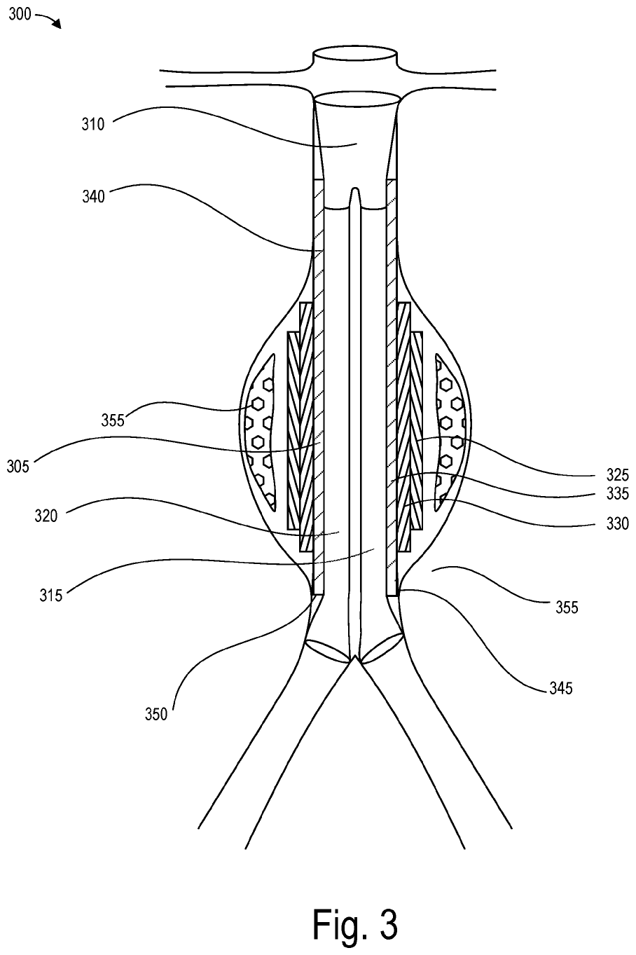

Referring to FIG. 3. FIG. 3 is an illustration 300 of a stent graft 305 surrounded by multiple layers of a stabilizing structure within the excluded aneurysm sac. The embodiment of the stabilizing structure shown in the illustration 300 comprises 3 flat layers that wrap around the stent graft 305. The 3 layers provide a continuous stabilizing force to maintain the stent graft 305 in its original position within the abdominal aortic aneurysm. Further, the 3 layers block the flow of blood into the excluded aneurysm sac 215 to either stop it or reduce the flow of blood, thus reducing the potential damage as a result of the endoleak.

The stent graft may comprise various configurations comprising various shapes and sizes. The disclosed stabilizing structure may be shaped to be configured to work with many types of stent grafts. The stent graft in the illustration 300 comprises a three-part structure including a main portion 310 that branches into a left bifurcated portion 315 and a right bifurcated portion 320. The stabilizing structure wraps around the three parts of the stent graft 305 and secures the stent graft 305 to a position within the aneurysm.

In various embodiments, a 1^{st} layer 325 wraps around the outside perimeter of the aneurysm. A 2^{nd} layer 330 wraps around the aneurysm just inside the 1^{st} layer 325. A 3^{rd} layer 335 wraps around the aneurysm just inside the 2^{nd} layer 330. The 1^{st} layer 325, 2^{nd} layer 330, and 3^{rd} layer 335 are positioned together to fit the shape of the excluded aneurysm sac 215.

As shown in the illustration 300, the 1^{st} layer 325 has a shorter length than the 2^{nd} layer as measured from the top of the illustration 300 to the bottom of the illustration 300. Similarly. the 2^{nd} layer has a shorter length than the 3^{rd} layer 335. The lengths of the three layers may be adjusted based on the shape of the aneurysm and the needs of the patient. But as the shape of most aneurysms is a lopsided spheroid, the lengths of the multiple sheets will tend to be similar to those shown in the illustration where the lengths are shorter for the layers on the outside perimeter of the aneurysm.

In addition to variable lengths, the widths of the 3 layers may also be adjusted based on the needs of the patient. For the purposes of this disclosure, the width of each layer refers to the distance as measured from the outside perimeter of the aneurysm to the stent graft 305 and in a direction normal to the surface of the stent graft 305. In various embodiments, the widths of the 1^{st} layer 325 and 2^{nd} layer 330 are thicker than the width of the 3^{rd} layer 335.

In various embodiments, the 3^{rd} layer 335 prevents blood leaks from access points by extending into an area 340 outside the aneurysm. The area outside the aneurysm, as used herein, refers to the area of the blood vessel that is not expanded to form the aneurysm. Thus, the portion of the 3^{rd} layer 335 that extends into the area 340 of the aortic artery, the area 345 of the left iliac artery, and the area 350 of the right iliac artery may aid in preventing type I endoleaks that leak blood from access points of the stent graft.

In various embodiments, the aneurysm includes one or more thrombi 355, which are also known as blood clots, deposited along the inner lumen of the aneurysm. The various layers of the stabilizing structure may be shaped to accommodate the thrombi 355. For instance, one or more layers of the stabilizing structure may be omitted because of the space taken by one or more thrombi. For instance, the stabilizing structure shown in FIG. 3 could be shaped to comprise 4 layers if the one or more thrombi 355 were not within the aneurysm.

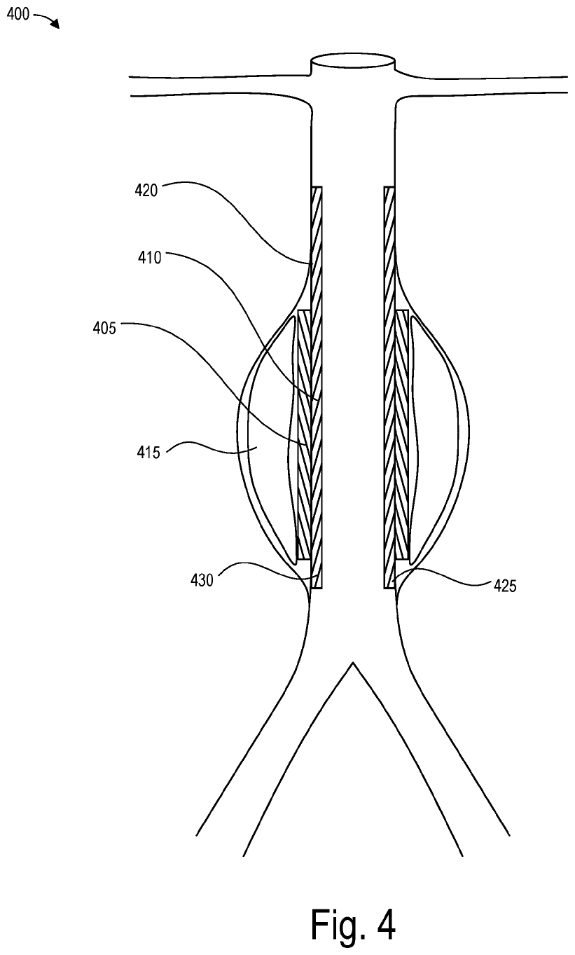

Referring to FIG. 4, FIG. 4 is an illustration 400 of a cross-section of multiple layers of a stabilizing structure and thrombi within the excluded aneurysm sac 215. The embodiment of the stabilizing structure shown in FIG. 4 comprises two layers. A 1^{st} layer is positioned on an outside perimeter of the excluded aneurysm sac 215. The thrombus 415 takes up a significant amount of space inside the aneurysm. Accordingly, the stabilizing structure is shaped around the thrombus 415 with the 1^{st} layer 405 making contact with the thrombus 415 and making contact with the inner lumen of the aneurysm. The 1^{st} layer 405 and 2^{nd} layer 410 are wrapped around the outer perimeter of the aneurysm and make a space for the stent graft.

The 2^{nd} layer 410 is positioned to the inside of the 1^{st} layer 405. The outside surface of the 2^{nd} layer 410 makes contact with the 1^{st} layer 405. Additionally, the 2^{nd} layer 410 makes contact with the inner lumen of the aortic artery at a position 420 upstream from the aneurysm and makes contact with the inner lumens of the left and right iliac arteries at position 425 and position 430 downstream from the aneurysm. The contact of the 2^{nd} layer at position 420. position 425, and position 430 provides pressure against the stent graft at the access points when the stent graft is deployed. Thus, the 2^{nd} layer 410 helps to mitigate type I endoleaks from the access points.

The 2^{nd} layer 410 may also be referred to as the inner layer of the stabilizing structure. The inner surface of the 2^{nd} layer 410 is configured to make contact with the outer surface of the stent graft when the stent graft is deployed to the aneurysm.

In various embodiments, the layers of the stabilizing structure comprise a resilient, clastic, and compressible material such as a foam. An exemplary embodiment of the material is a biocompatible foam such as a polycarbonate polyurethane-urea foam described in U.S. Pat. Nos. 7,803,395 and 8,337,487, which are incorporated by reference in their entirety. The polycarbonate polyurethane-urea foam may comprise different formulations whereby properties such as average cell size, density. permeability, compressive strength, tensile strength parallel, tensile strength perpendicular, elongation parallel, and elongation perpendicular are modified.

For instance, various formulations of the polycarbonate polyurethane-urea foam may have an average cell size that varies from about 250 μm to about 650 μm. The various formulations of the polycarbonate polyurethane-urea foam may have a density that varies from about 2.5 lb/ft³ to about 7.0 lb/ft³. The various formulations of the polycarbonate polyurethane-urea foam may have a permeability that varies from about 70 Darcy to about 350 Darcy. The various formulations of the polycarbonate polyurethane-urea foam may have a compressive strength that varies from about 0.9 psi to about 4.5 psi. The various formulations of the polycarbonate polyurethane-urea foam may have a tensile strength parallel that varies from about 60 psi to about 90 psi. The various formulations of the polycarbonate polyurethane-urea foam may have a tensile strength perpendicular that varies from about 25 psi to about 75 psi. The various formulations of the polycarbonate polyurethane-urea foam may have an elongation parallel that varies from about 120% to about 300%. The various formulations of the polycarbonate polyurethane-urea foam may have an elongation perpendicular that varies from about 120% to about 300%.

The stabilizing structure may comprise various foams such as biocompatible foams. Various advantages of biocompatible foams such as polycarbonate polyurethane-urea foam include. but are not limited to being non-cytotoxic, having weak allergic potential, being a negligible irritant, being non-toxic, being non-mutagenic, being non-clastogenic, and being non-hemolytic. Various polycarbonate polyurethane-urea foams do not react with intramuscular implants up to 12 weeks after implantation. Neurological implants made of various polycarbonate polyurethane-urea foams are well-tolerated in rabbits up to 24 weeks after implantation and show no local or systemic signs of toxicity. Studies in dogs show no significant amounts of thrombosis subsequent to implantation with various polycarbonate polyurethane-urea foams. The various polycarbonate polyurethane-urea foams are non-pyrogenic.

Various polycarbonate polyurethane-urea foams have been shown to support progressive healing that is characterized by rapid fibrovascular tissue ingrowth. Further, vascularization has been observed through the porous foam structure. Complete biointegration without encapsulation has also been shown. Clinical studies have shown reduced pain and reduced recurrence rates after when using a polycarbonate polyurethane-urea foam compared to conventional procedures.

The layers of the stabilizing structure may be delivered to the location of the aneurysm in a compressed state within a catheter. In an exemplary embodiment, the layers of the stabilizing structure may expand without applying any force once they are deployed from the catheter. The layers may expand to their original uncompressed shape or until they make contact with objects inside the aneurysm such as the inner lumen of the aneurysm, a thrombus, another layer, and stent graft. or any other medical devices.

In cases where the layers expand to make contact with another object, the layers may be at least partially compressed and may apply a static force to objects with which they make contact. The static force delivered by each of the layers of the stabilizing structure may help to stabilize a stent graft that is positioned within the innermost layer of the stabilizing structure. Further, the inner layer of the stabilizing structure may similarly provide a static force to the access points at position 420, position 425, and position 430, which may stabilize the stent graft to the access point and provide a tighter seal from blood entering the excluded aneurysm sac.

The stabilizing structure may comprise various numbers of layers depending on the needs of the patient and shape of the aneurysm. The embodiment of the stabilizing structure shown in FIG. 4 has 2 layers, a 1^{st} layer 405 and a 2^{nd} layer 410. Various embodiments of the stabilizing structure may comprise fewer or more layers. For example, a stabilizing structure may comprise 1 layer, 2 layers, 3 layers, 4 layers, 5 layers, or 6 layers. Further, and although not shown in the figures, the length of each of the layers may not be constant around the entire layer. For instance, a layer may have a variable length that tapers longer or shorter based on the dimensions of the aneurysm. The length of the layers may be adjusted based on objects within the aneurysm such as a thrombus or medical devices.

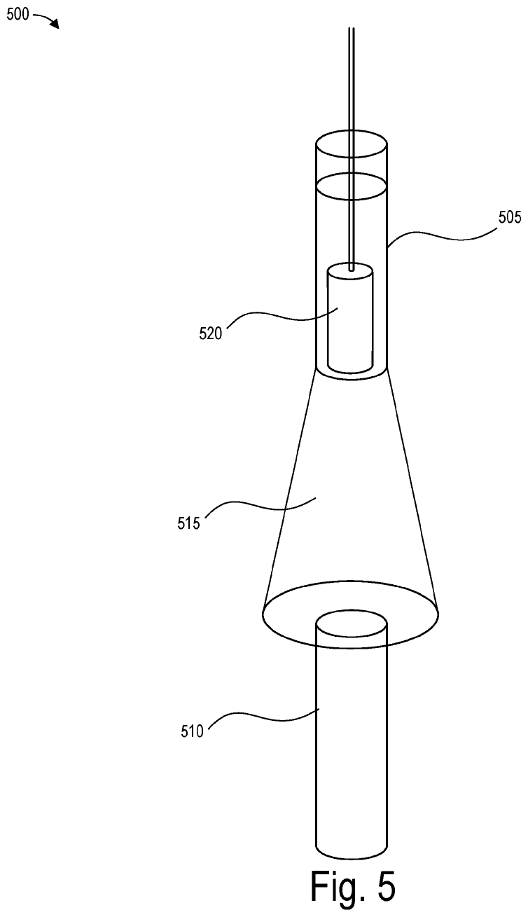

Referring to FIG. 5, FIG. 5 is an illustration 500 of a delivery system for delivering the stabilizing structure 510 to an aneurysm. In various embodiments, the delivery system may comprise one or more catheters 505 that deliver the stabilizing structure to a deployment location in a blood vessel. The stabilizing structure 510 may be delivered while in a compressed state that takes up a small volume.

Once the catheter 505 brings the stabilizing structure to the delivery location, the stabilizing structure may be extracted from the catheter by various means. In an exemplary embodiment, the layers of the stabilizing structure 510 are pushed out of the catheter 505 with a plunger 520. The one or more layers of the stabilizing structure 510 may be delivered by the same catheter 505 in one delivery, by the same catheter 505 in separate deliveries, or by separate catheters 505.

In various embodiments, the catheter 505 includes a dilator 515 at an end of the catheter 505 from which the one or more layers of the stabilizing structure 510 are exited from the catheter 505. As shown in the illustration 500, the dilator 515 may comprise a conelike shape with a proximal end of the dilator 515 attached to an end of the catheter 505 and a distal end of the dilator 515 that is configured to allow the one or more layers of the stabilizing structure 510 to be pushed out into a blood vessel.

In the exemplary embodiment shown in FIG. 5, the diameter of the distal end of the dilator 515 is greater than the diameter of the proximal end of the dilator 515. The proximal end, which is attached to an end of the catheter 505, has the same diameter as the end of the catheter 505 to which it is attached. The gradual expansion of the diameter of the dilator 515, from its proximal end to its distal end, allows the stabilizing structure to gradually expand as it is pushed through the dilator 515 by the plunger 520. Accordingly, the dilator 515 facilitates the deployment of the one or more layers of the stabilizing structure 510.

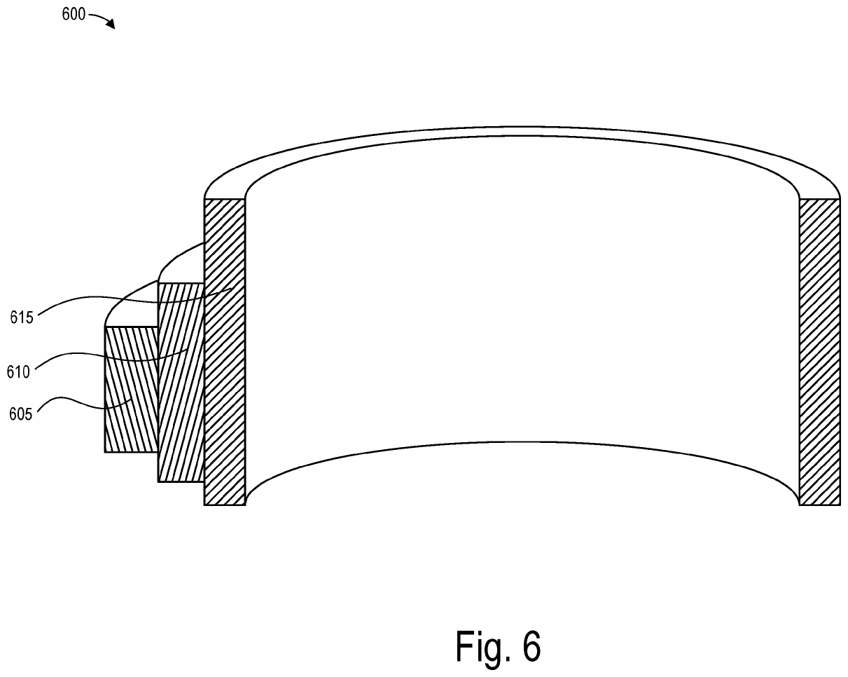

Referring to FIG. 6, FIG. 6 is an illustration 600 of a tapered layering of the stabilizing structure without environmental features such as an aneurysm, blood vessels, or a stent graft. In various embodiments, the stabilizing structure comprises two or more layers of material that are configured to at least partially surround a stent graft. The multiple layers of the stabilizing structure may be shaped to conform to the space inside an aneurysm.

Accordingly, a 1^{st} layer 605, which is positioned on an outside layer of the stabilizing structure, may have a height, measured from the top of the illustration 600 to the bottom of the illustration 600, that is smaller than the height of the other layers. The 2^{nd} layer 610, which is positioned in the middle layer of the stabilizing structure. has a height that is gradually bigger than the height of the 1^{st} layer 605 and smaller than the height of the 3^{rd} layer 615. The 3^{rd} layer 615 is positioned as the inside layer of the stabilizing structure. The 3^{rd} layer 615 has the greatest height of the layers. In various embodiments, such as the embodiment shown in FIG. 3, the 3^{rd} layer 615 extends out of the aneurism portion of the blood vessel. Thus, the stabilizing structure may partially extend out of the aneurysm.

In various embodiments, the various layers of the stabilizing structure are deployed in series. In one example, the 1^{st} layer 605 is deployed from the catheter 505 before the other layers and allowed to expand within the aneurysmal sac. The 2^{nd} layer 610 is deployed next and positioned to expand inside of the 1^{st} layer 605. The 3^{rd} layer 615 is deployed after the 2^{nd} layer 610 and allowed to expand into the 2^{nd} layer 610.

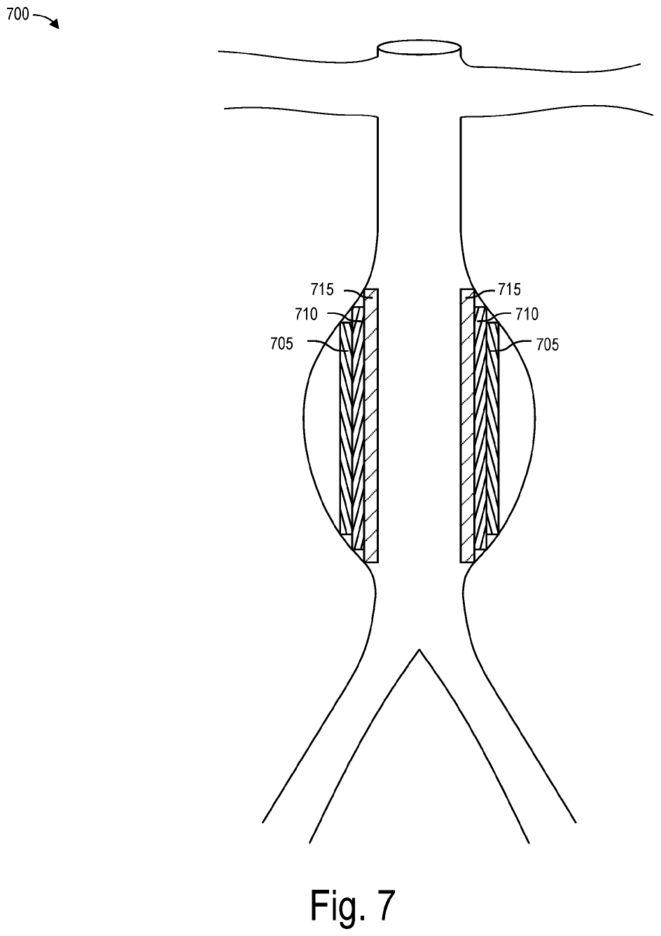

Referring to FIG. 7, FIG. 7 is an illustration 700 of the tapered layering of the stabilizing structure after being deployed in an aneurysm and before deployment of a stent graft. In various embodiments, the stabilizing structure is configured to be deployed before deployment of a stent graft. Accordingly, the stabilizing structure is shaped to fill the space that will become the excluded aneurysm sac subsequent to deployment of the stent graft.

In various embodiments, the stabilizing structure comprises multiple layers of material that wrap around an outer perimeter of an aneurysm. The number of layers may vary depending on multiple factors including but not limited to a shape of the aneurysm, the medical need, and material of the stabilizing structure. In an exemplary embodiment, the outside layers of the stabilizing structure are deployed first. Thus, for the three layered stabilizing structure shown in the illustration 700, the 1^{st} layer 705 is deployed first. followed by deployment of the 2^{nd} layer 710. and then followed by deployment of the 3^{rd} layer 715.

Depending on the shape and objects such as thrombi in the aneurysm, the outside of the 1^{st} layer 705 may be in contact with the inner lumen of the aneurysm. Thus, the material for the 1^{st} layer 705 may be configured to be in contact with the inner lumen in a way that is different from the other layers. For instance, the 1^{st} layer 705 may be softer than the other layers, allowing it to better conform to the irregular surface of the aneurysm's inner lumen. In various embodiments, the 1^{st} layer 705 may have a curved outer surface to better conform to the inner lumen of the aneurysm.

Likewise, the inner surface of the 3^{rd} layer 715, which may be brought into contact with a stent graft, may be configured for that purpose. In various embodiments, the material of the 3^{rd} layer 715 may be configured to allow the stent graft to slide past it during a deployment of the stent graft. In various embodiments, the 3^{rd} layer 715 may be more rigid or softer than the other layers based on the specific needs of the patient. For instance, the 3^{rd} layer 715 may be configured to be more rigid than the other layers so as to resist re-positioning of the stent graft after deployment.

The shape of the 3^{rd} layer 715 may also be configured to facilitate deployment of the stent graft. For example, the stent graft may be guided to one or more positions that are marked by the 3^{rd} layer 715. In one instance, the 3^{rd} layer 715 is marked with radiopaque materials to aid a medical practitioner in guiding and deploying the stent graft with the aneurysm.

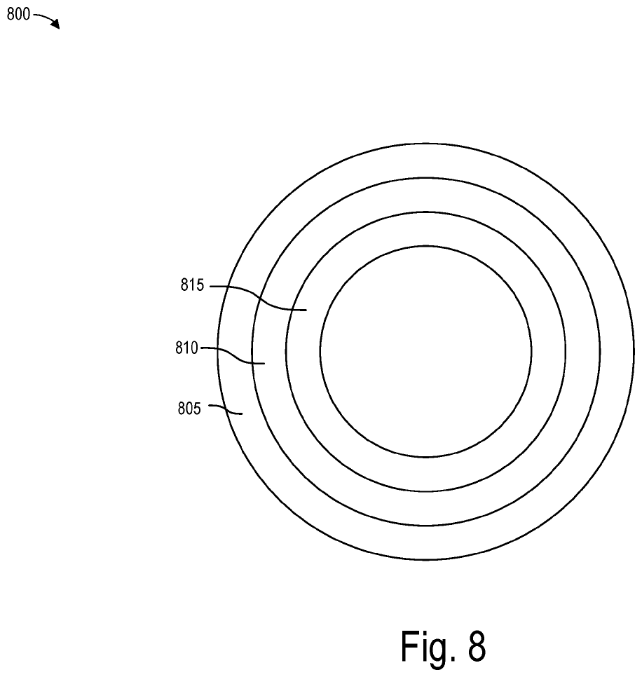

Referring to FIG. 8, FIG. 8 is a photograph 800 of multiple layers of a stabilizing structure from a top view. The stabilizing structure may be configured to be deployed in a configuration of a cylinder with multiple concentric rings, as shown in the photograph 800. The concentric rings in the photograph comprise a 1^{st} layer 805, a 2^{nd} layer 810, and a 3^{rd} layer 815.

The 1^{st} layer 805, a 2^{nd} layer 810, and a 3^{rd} layer 815 may each comprise a flat sheet of material that is wrapped into a cylinder. In various embodiments, the sheets may be deployed in a wrapped configuration whereby the sheets are locked into the cylinder shape prior to deployment. Alternatively, the sheets may be deployed prior to being wrapped into a cylinder shape. For instance, a sheet may be deployed as a flat sheet and not rolled into a cylinder. After deployment of the flat sheet, the medical practitioner may position the sheet to wrap around the perimeter of an aneurysm.

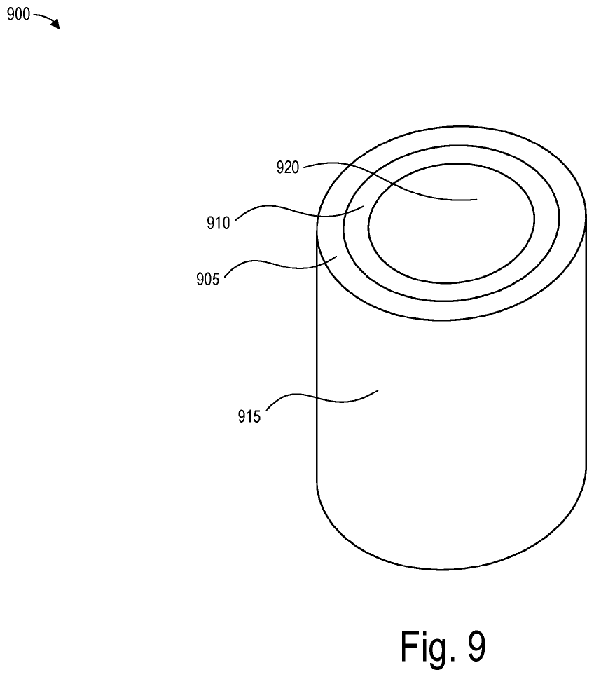

Referring to FIG. 9, FIG. 9 is a photograph 900 of multiple layers of a stabilizing structure from a top perspective view. The multiple layers in the photograph arc wrapped to form a cylinder. The embodiments of the stabilizing structure shown in FIGS. 8 and 9 show layers with the same height, as measured from one end of the cylinder to the other end. In various other embodiments, such as those depicted in FIG. 5, the sheets have variable heights that decrease for each layer starting from the innermost layer.

The photograph shows two layers, a 1^{st} layer 905 and a 2^{nd} layer 910, configured into a hollow cylinder. The 1^{st} layer 905 comprises an outer ring of the cylinder and the 2^{nd} layer 910 comprises an inner ring of the cylinder. In various embodiments, the outer side 915 of the 1^{st} layer 905, which is the side of the 1^{st} layer 905 that comprises the outer side of the cylinder, may be configured to contact the walls of a blood vessel. Accordingly. the outer side 915 of the 1^{st} layer may comprise a surface that will not damage the lumen of a blood vessel. In an exemplary embodiment, the surface on the outer side 915 is textured to latch onto the inner lumen of a blood vessel and mitigate slipping of the stabilizing structure relative to the blood vessel.

The inner side 920 of the 2^{nd} layer 910 comprises the inner side of the hollow cylinder. The surface of the inner side 920 may be configured to make contact with the stent graft. For instance, the inner side 920 may be textured to conform to a surface on the stent graft. In various embodiments, the inner side 920 may be configured with a soft material that reduces a risk of tearing in a stent graft and thus mitigates the risk of a type III endoleak.

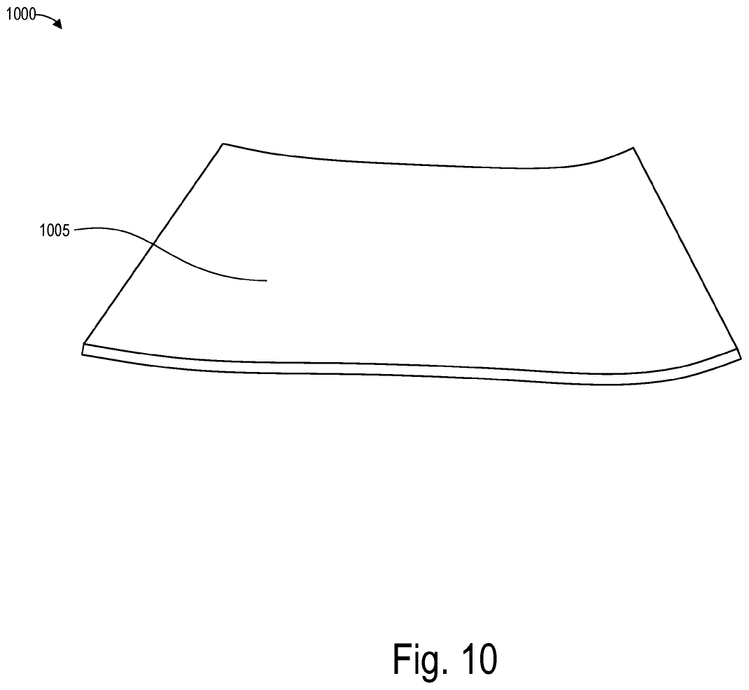

Referring to FIG. 10, FIG. 10 is a photograph 1000 of a sheet of material used as the stabilizing structure from a perspective view. The layers, which may be configured to wrap around a stent graft, may be produced as a flat sheet 1005 that is later wrapped into a cylinder. In various embodiments, the flat sheets are configured to not completely wrap around a stent graft and only form partial cylinders inside the aneurysm.

Once produced, the flat sheet 1005 may be trimmed to configure its size for deployment. For example, a 1^{st} layer may be configured to be positioned on an outside layer of a stabilizing structure such as the 1^{st} layer 325 shown in FIG. 3. Accordingly, the 1^{st} layer may be trimmed to have a shorter length than other layers. Similarly, the width of the 1^{st} layer, which is determined by the circumference of the cylinder, may be trimmed to be longer than the other layers. The other layers, such as a 2^{nd} layer or a 3^{rd} layer, may be similarly trimmed according to the shape and dimensions of the stabilizing structure.

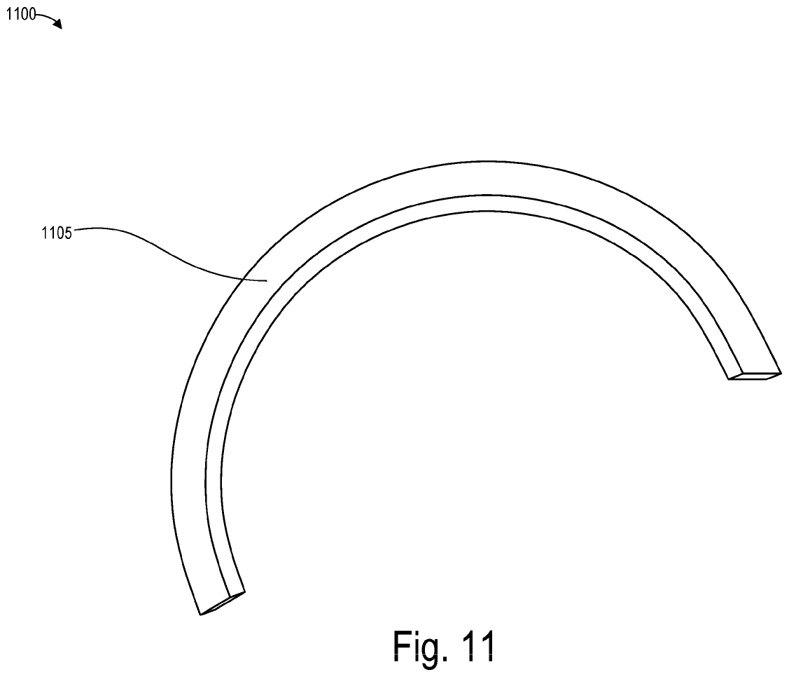

Referring to FIG. 11, FIG. 11 is a photograph 1100 of a single layer 1105 of a curved material used as the stabilizing structure from a top view. The layers, which are deployed to wrap around the stent graft, may be manufactured to have a naturally curved shape. Thus, the layer 1105 may not produce any forces that cause it to bend back into a flat shape. Further, deployment of the naturally curved sheet, which does not wrap all the way around into a cylinder, may be easier using materials such as those shown in FIG. 11 that are naturally curved.

In various embodiments, two or more sheets may be separately deployed for a single layer. For example, two sheets that each comprise a semi-circle may be deployed separately and positioned to form a single layer of the stabilizing structure.

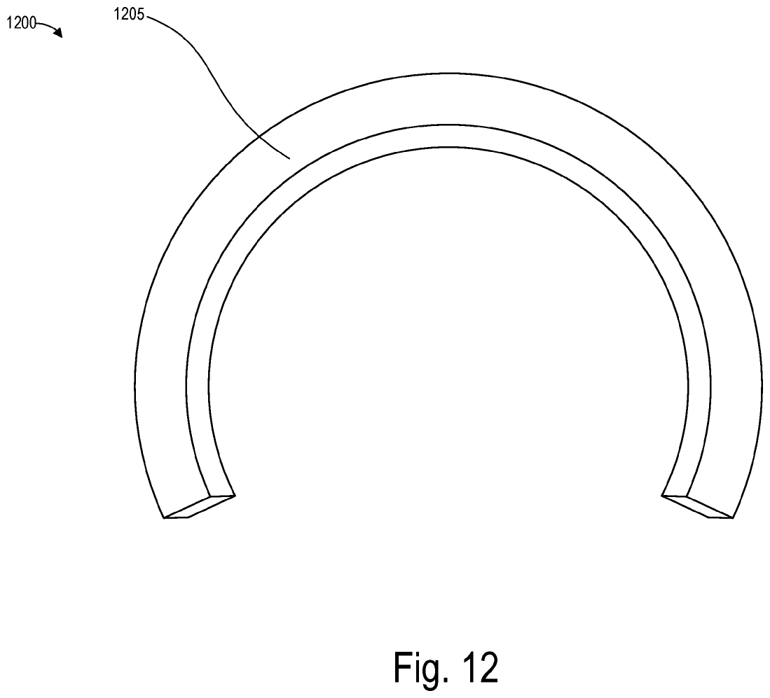

Referring to FIG. 12, FIG. 12 is a photograph 1200 of a sheet 1205 of a curved material used as the stabilizing structure from a top view. In various embodiments, the sheet 1205 may or may not be wrapped into a cylinder prior to deployment. In an exemplary embodiment, the sheet 1205 is deployed as shown in FIG. 12 without wrapping the sheet 1205 into a cylinder prior to deployment. The empty space, which would be left by the sheet not wrapping all the way around, may accommodate a unique shape of an aneurysm or accommodate one or more other objects within the aneurysm.

As the sheet is deployed in an aneurysm, the sheet may be manipulated by a practitioner to a desired position. In various embodiments, the sheet 1205 may be deployed after deployment of one or more medical devices within the aneurysm. Thus, the sheet 1205 could be positioned to wrap around the one or more objects that were deployed before the sheet 1205.

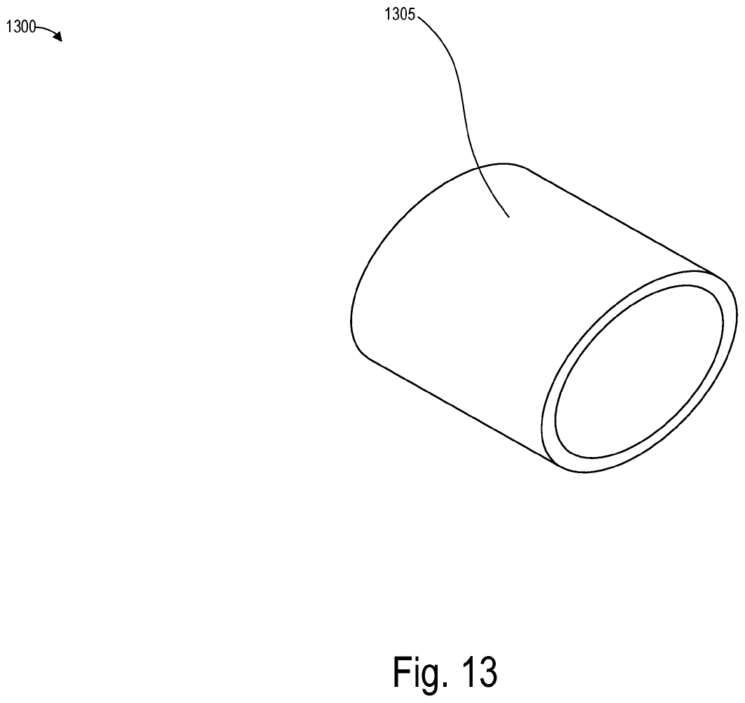

Referring to FIG. 13, FIG. 13 is a photograph 1300 of a layer 1305 of a circular portion of the stabilizing structure from a perspective view. The layer 1305 is shown in an uncompressed state. Prior to deployment, the layer 1305 may be compressed within the catheter 505 to be transported to a location of an aneurysm. And after being exited from the catheter, the layer 1305 may, at least partially, expand toward its original shape shown in the photograph 1300.

As the layer 1305 expands after being extracted by any means from the catheter, it may not have space to fully expand back to its original uncompressed state. Thus, the layer 1305 would continually exert a force directed outward from the outer surface of the layer 1305 as the layer 1305 is confined within the space that is smaller than its uncompressed volume. Accordingly, the layer 1305 will effectively secure itself in place subsequent to deployment.

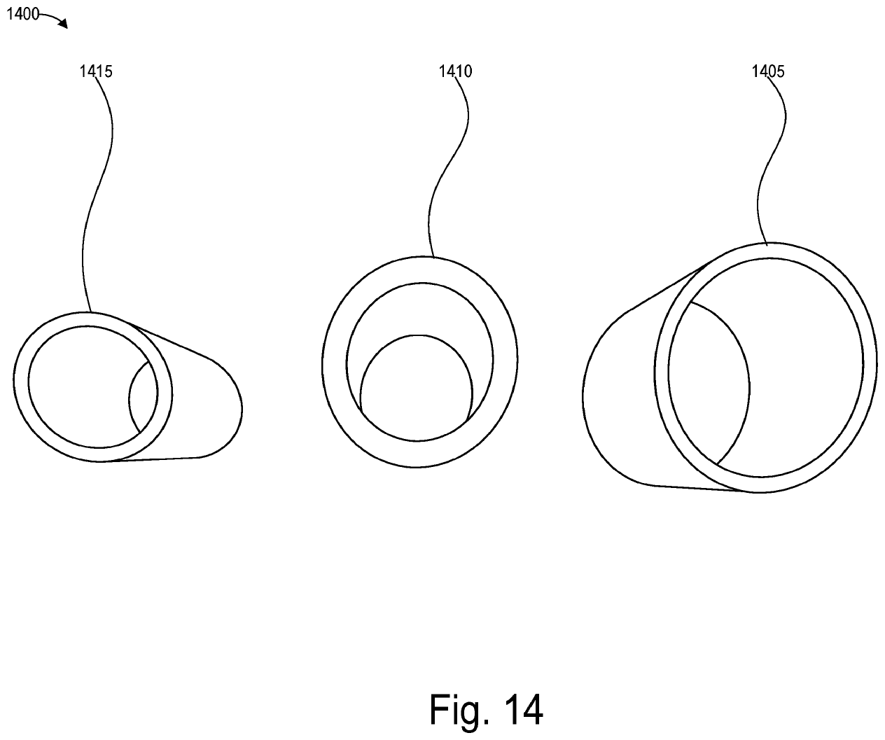

Referring to FIG. 14, FIG. 14 is a photograph 1400 of multiple layers of a stabilizing structure apart from one another from a top view. In various embodiments. each of the multiple layers may be deployed separately. For example, a 1^{st} layer 1405 may be deployed first and allowed to expand within an aneurysm. Then. a 2^{nd} layer 1410 may be deployed within the 1^{st} layer 1405 and allowed to expand and make contact with the 1^{st} layer 1405. Likewise, a 3^{rd} layer 1415, which is the smallest diameter cylinder in the photograph, may be deployed within the 2^{nd} layer 1410 and allowed to expand to make contact with the 2^{nd} layer 1410.

In various embodiments, the multiple layers may be assembled into a multilayered stabilizing structure before deployment into an aneurysm. The pre-assembled stabilizing structure would be more massive than separate layers, but would be faster and simpler to deploy than separately deploying the multiple layers.

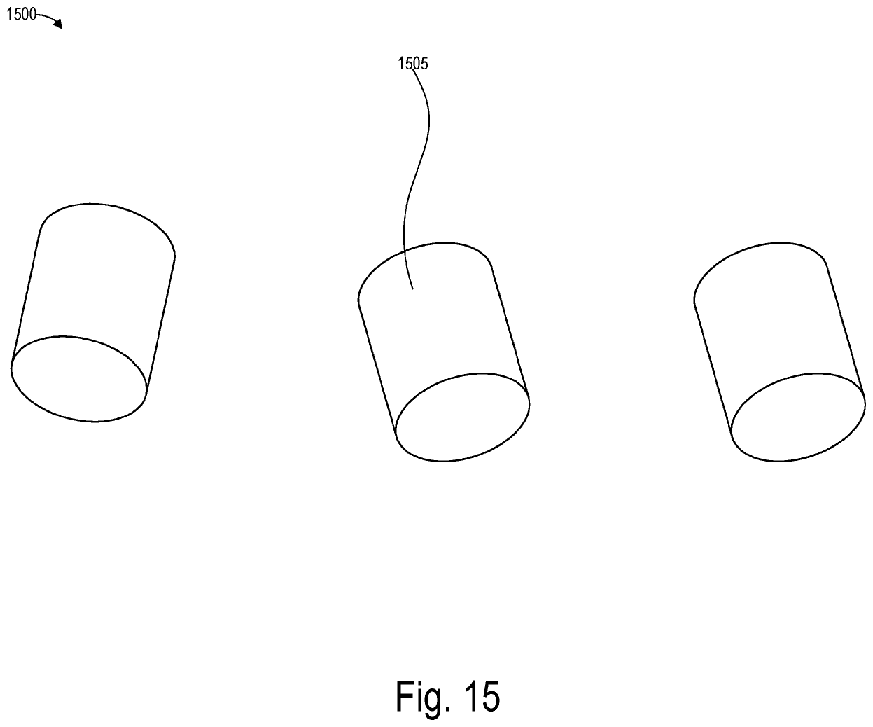

Referring to FIG. 15, FIG. 15 is a photograph 1500 of an embodiment of the stabilizing structure comprising pellets 1505 of material to be inserted into the excluded aneurysm sac. The material may comprise a resilient compressible material such as a foam. The pellets may be referred to as longitudinal elements as they have a cylindrical shape. In various embodiments, the pellets, or longitudinal elements, may be compressed along their longitudinal axis, which runs the ends of their cylindrical shapes. The pellets may be used in combination with the sheets of material such as those shown in FIG. 14.

The pellets 1505 may be deployed into an aneurysm to occupy space that a bigger structure such as the sheet of material shown in FIG. 14 could not occupy. In various embodiments, the pellets 1505 may be deployed into irregularly shaped portions of an aneurysm.

In various embodiments, the pellets 1505 are deployed subsequent to deployment of a stent graft. Accordingly, the pellets 1505 may be utilized to repair a stent graft for a patient that is experiencing an endoleak. The pellets 1505 may be used in response to any type of endoleak. The pellets 1505 may approach the stabilizing structure via the catheter 505 from any one of the iliac arteries or the aortic artery. Once at the position of the stent graft, the pellets 1505 may be forced into the space between the stent graft and the inner lumen of the artery.

In an exemplary embodiment. the pellets 1505 may be deployed in response to a type I endoleak where blood is flowing into the excluded aneurysm sac 215 through one of the access points of the stent graft. In one example of repairing a type I endoleak, the pellets 1505 may be forced in between the stent graft and inner lumen of the blood vessel at any of the access points of the stent graft. In some instances, the leak point at one of the access points of the stent graft may be identified, whereby blood enters the excluded aneurysm sac 215 through the leak point. The pellets 1505 may be forced into the leak point to repair the type I endoleak by either stopping the leak or mitigating the leak such that it is less dangerous.

Similarly. the pellets 1505 may be used to repair other types of endoleaks. For instance. the pellets 1505 may be used to repair a type III endoleak where blood leaks through a defect in the stent graft. In one example, the pellets 1505 may be inserted into the defect of the stent graft to stop or reduce the flow of blood into the excluded aneurysm sac 215. In various embodiments, the pellets 1505 may be inserted into the excluded aneurysm sac 215 to generally slow a flow of blood into the excluded aneurysm sac 215.

In various embodiments, the pellets 1505 are deployed into an aneurysm prior to deployment of the cylindrical sheets of the stabilizing structure. Alternatively, the pellets 1505 may be deployed subsequent to deployment of the cylindrical sheets. For instance, the pellets 1505 may be deployed responsive to determining that the stabilizing structure is not secure. The pellets 1505 could then be added to create less space in the aneurysm and thus make the stabilizing structure more secure.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. A system for treating an aneurysm, the system comprising:
a stabilizing structure that is configured to surround a lumen structure that facilitates blood flow through the aneurysm;
the stabilizing structure comprising one or more elements that are configured to expand to fill a space in an aneurysmal sac.

2. The system of claim 1, wherein at least one of the one or more elements comprises a sheet that at least partially wraps around the lumen structure.

3. The system of claim 1, wherein at least two of the one or more elements comprises a sheet or a tube that at least partially wraps around the lumen structure; and
wherein the at least two of the one or more elements wrap around the lumen structure in layers.

4. The system of claim 3, wherein lengths of the stabilizing structure, as measured along a flow of blood in the lumen structure, of two or more elements are tapered such that the lengths decrease from an inside layer to an outside layer.

5. The system of claim 4, wherein tapering of the layers is configured to fit within the space in the aneurysmal sac.

6. The system of claim 1, wherein at least one of the one or more elements comprise a longitudinal clement.

7. The system of claim 3, wherein at least one of the one or more elements further comprise a longitudinal element; and
wherein the longitudinal element is configured to be inserted in a space of the aneurysmal sac outside the at least one sheet or a tube that at least partially wraps around the lumen structure.

8. The system of claim 7, wherein the stabilizing structure comprises a multitude of longitudinal elements.

9. The system of claim 5, wherein the stabilizing structure prevents displacement of the lumen structure within the aneurysm.

10. The system of claim 5, wherein the stabilizing structure lowers a risk of an endoleak compared to the lumen structure without the stabilizing structure.

11. The system of claim 10, wherein the endoleak comprises a type I, type II, type III, or type IV endoleak.

12. The system of claim 1, wherein the one or more elements comprise a foam material.

13. The system of claim 4, wherein the one or more elements are configured to be delivered to the aneurysmal sac prior to delivery of the lumen structure.

14. The system of claim 13, wherein sheets or tubes are configured to be delivered in an order from an outermost layer to an innermost layer.

15. A support structure for a stent graft, the support structure comprising:
one or more elements that are configured to expand to fill a space in an aneurysmal sac;
at least one of the one or more elements comprises a sheet or tube that at least partially wraps around the stent graft.

16. The support structure of claim 15, wherein the one or more elements concentrically wrap around the stent graft in layers;
wherein the one or more elements comprise at least two elements;
wherein lengths of at least two elements, as measured along a flow of blood in the aneurysmal sac, are tapered such that the lengths decrease from an inside layer to an outside layer;
wherein the at least two elements are configured to be delivered in an order from an outermost layer to an innermost layer; and
wherein at least two elements are configured to be delivered prior to delivery of the stent graft.

17. A system for treating an aneurysm, the system comprising:
an endograft comprising an interior lumen that facilitates blood flow across an aneurysmal sac;
one or more elements that are configured to expand to fill a space in the aneurysmal sac between the endograft and interior walls of the aneurysmal sac;
at least one of the one or more elements comprise sheets or tubes that at least partially wrap around the endograft.

18. The system of claim 17, wherein the one or more elements wrap around the endograft in layers; and
wherein the one or more elements are configured to be delivered to the aneurysmal sac in an order from an outermost layer to an innermost layer.

19. The system of claim 18, wherein lengths, as measured along a flow of blood in the interior lumen, of the one or more elements are tapered such that the lengths decrease from an inside layer to an outside layer.

20. The system of claim 19, wherein the one or more elements are configured to be delivered through an opening of a catheter that comprises a dilator.
